(19) United States
(12) Patent Application Publication
(10) Pub. No.: US 2024/0065581 A1
(43) Pub. Date: Feb. 29, 2024

(54) GAIT MEASUREMENT SYSTEM, GAIT MEASUREMENT METHOD, AND PROGRAM RECORDING MEDIUM

(71) Applicant: NEC Corporation, Minato-ku, Tokyo (JP)

(72) Inventors: Chenhui HUANG, Tokyo (JP); Kenichiro FUKUSHI, Tokyo (JP); Zhenwei WANG, Tokyo (JP)

(73) Assignee: NEC Corporation, Minato-ku, Tokyo (JP)

(21) Appl. No.: 17/766,807

(22) PCT Filed: Oct. 29, 2019
(86) PCT No.: PCT/JP2019/042360

§ 371 Date: Apr. 6, 2022

(51) Int. Cl. A61B 5/11; A61B 5/00; G16H 50/20

(52) U.S. Cl. CPC A61B 5/112; A61B 5/7235; A61B 5/742; G16H 50/20

(57) ABSTRACT

In order to provide a gait measurement system by which gait symmetry in day-to-day life can easily be measured, the present invention offers a gait measurement system comprising a data acquisition device that measures physical quantities associated with the respective movement of the left and right feet, and a calculation device that uses the physical quantities associated with the respective movement of the left and right feet to calculate gait symmetry.

### TECHNICAL FIELD

The present invention relates to a gait measurement system, a gait measurement method, and a program. In particular, the present invention relates to a gait measurement system for measuring a symmetry of walking, a gait measurement method, and a program.

### BACKGROUND ART

With increasing interest in healthcare for physical condition management, a technique for measuring a gait including features of walking of a pedestrian has been developed.

PTL 1 discloses a walking change determination device that includes an acceleration sensor and determines a change in walking of a user based on detected acceleration. The device of PTL 1 determines a degree of change, which is a degree of temporal change, based on the acceleration detected by the acceleration sensor and based on a temporal change in a trajectory during walking of a predetermined region to which the device is attached.

PTL 2 discloses a walking analysis system that calculates a stride length of a pedestrian using measurement data of sensors attached to a back of a foot, a lower leg, and a upper thigh of at least one of left and right feet of the pedestrian.

PTL 3 discloses a walking analysis device that performs walking analysis of a subject using a body-worn sensor having a triaxial angular velocity sensor attached to each of a plurality of body parts including lower limbs of the subject.

### CITATION LIST

### Patent Literature

### [PTL 1] JP 5724237 B

### [PTL 2] JP 5586050 B

### [PTL 3] JP 2018-015017 A

### Non Patent Literature

[NPL 1] S. Madgwick, A. Harrison, R. Vaidyanathan, “Estimation of IMU and MARG orientation using a gradient descent algorithm,” 2011 IEEE International Conference on Rehabilitation Robotics, Rehab Week Zurich, ETH Zurich Science City, Switzerland, June 29-July 1, pp. 179-185, 2011.
[NPL 2] Y. Morio, et al, “The Relationship between Walking Speed and Step Length in Older Aged Patients,” Diseases 2019, 7, 17.

### SUMMARY OF INVENTION

### Technical Problem

When the device of PTL 1 is attached to a waist of a pedestrian, stride lengths of the left and right feet of the pedestrian can be calculated by specifying the positions of the feet from the projection of the measured waveform. However, in the method of PTL 1, since the stride length cannot be accurately calculated unless the lower limb is in a straight state, the accuracy is deteriorated in case where an ankle joint is distorted.

According to the method of PTL 2, the sensor unit is attached to both feet, and waveforms of both feet can be measured by synchronizing measurement data of both feet. However, in the method of PTL 2, it is necessary to attach sensors to a plurality of positions on both feet, and thus, it is difficult to use the method on a daily basis.

According to the method of PTL 3, it is possible to remove a drift error from attitude angles of each axis of the sensor obtained from the measurement value of the triaxial angular velocity sensor and quantify a difference between left and right walking events. However, in the method of PTL 3, it is necessary to attach sensors to a plurality of body parts including lower limbs of a subject, and thus, it is difficult to use the method on a daily basis.

It is important for healthcare to detect abnormality of walking of a pedestrian that affect measured data such as a stride length. From the viewpoint of abnormal detection of the walking, for example, there is a need to measure a symmetry of walking of a pedestrian as the gait of the pedestrian. When the symmetry of walking can be measured in real time, the abnormality occurring in a pedestrian can be found at an early stage. Therefore, a technique for measuring the symmetry of walking in day-to-day life is required. However, PTLs 1 to 3 do not disclose such a technique.

An object of the present invention is to solve the above-described problems and to provide a gait measurement system and the like capable of easily measuring a symmetry of walking in day-to-day life.

### Solution to Problem

According to an aspect of the present invention, a gait measurement system includes a data acquisition device configured to measure physical quantities related to movements of both left and right feet, and a calculation device configured to calculate a symmetry of walking using the physical quantities related to the movements of the left and right feet.

In a gait measurement method according to an aspect of the present invention, a computer acquires physical quantities related to movements of left and right feet, and calculates a symmetry of walking using the acquired physical quantities related to the movements of the left and right feet.

According to an aspect of the present invention, a program causes a computer to perform a process including acquiring physical quantities related to movements of left and right feet and calculating a symmetry of walking using the acquired physical quantities related to the movements of the left and right feet.

### Advantageous Effects of Invention

According to the present invention, it is possible to provide a gait measurement system and the like capable of easily measuring the symmetry of walking in day-to-day life.

### BRIEF DESCRIPTION OF DRAWINGS

FIG. 1 is a block diagram illustrating an example of a configuration of a gait measurement system according to a first example embodiment of the present invention.

FIG. 2 is a conceptual diagram illustrating an arrangement example of a data acquisition device of the gait measurement system according to the first example embodiment of the present invention.

FIG. 3 is a conceptual diagram for describing a coordinate system of sensor data acquired by the gait measurement system according to the first example embodiment of the present invention.

FIG. 4 is a conceptual diagram for describing an example of a walking parameter used by the gait measurement system according to the first example embodiment of the present invention.

FIG. 5 is a conceptual diagram for describing another example of the walking parameter used by the gait measurement system according to the first example embodiment of the present invention.

FIG. 6 is a conceptual diagram for describing another example of the walking parameter used by the gait measurement system according to the first example embodiment of the present invention.

FIG. 7 is a conceptual diagram for describing a coordinate system of an attitude angle calculated by the gait measurement system according to the first example embodiment of the present invention.

FIG. 8 is a graph for describing time-series data of the attitude angle generated by the gait measurement system according to the first example embodiment of the present invention.

FIG. 9 is a graph for describing time-series data of a sensor height generated by the gait measurement system according to the first example embodiment of the present invention.

FIG. 10 is a block diagram illustrating an example of a configuration of the data acquisition device of the gait measurement system according to the first example embodiment of the present invention.

FIG. 11 is a block diagram illustrating an example of a configuration of a calculation device of the gait measurement system according to the first example embodiment of the present invention.

FIG. 12 is a flowchart for describing an example of an operation of a walking parameter calculation unit of the calculation device of the gait measurement system according to the first example embodiment of the present invention.

FIG. 13 is a flowchart for describing an example of an operation of a symmetry calculation unit of the calculation device of the gait measurement system according to the first example embodiment of the present invention.

FIG. 14 is a block diagram for describing an example of a configuration of a calculation device of a gait measurement system according to a second example embodiment of the present invention.

FIG. 15 is a block diagram illustrating an example of a configuration of the calculation device of the gait measurement system according to the second example embodiment of the present invention.

FIG. 16 is a conceptual diagram for describing a position of marks attached to a periphery of a shoe when generating a regression model used by the gait measurement system according to the second example embodiment of the present invention.

FIG. 17 is a conceptual diagram for describing a walking line on which a pedestrian walks when generating the regression model used by the gait measurement system according to the second example embodiment of the present invention, and for describing an arrangement of a plurality of cameras for detecting the walking of the pedestrian.

FIG. 18 is a diagram illustrating a measurement result showing a relationship between a symmetry of attitude angles and a symmetry of step lengths generated with respect to walking of two subjects.

FIG. 19 is a diagram illustrating a measurement result showing a relationship between a symmetry of sensor heights and the symmetry of the step lengths generated with respect to the walking of two subjects.

FIG. 20 is a flowchart for describing an example of an operation of a step length calculation unit of the gait measurement system according to the second example embodiment of the present invention.

FIG. 21 is a block diagram for describing an example of a configuration of a gait measurement system according to a third example embodiment of the present invention.

FIG. 22 is a conceptual diagram illustrating an example of information to be displayed on a display unit of a display device of the gait measurement system according to the third example embodiment of the present invention.

FIG. 23 is a flowchart for describing an example of an operation of the gait measurement system according to the third example embodiment of the present invention.

FIG. 24 is a conceptual diagram illustrating an example of a configuration of a gait measurement system according to a modified example of the third example embodiment of the present invention.

FIG. 25 is a conceptual diagram illustrating an example of information to be displayed on a display unit of a display device of the gait measurement system according to the modified example of the third example embodiment of the present invention.

FIG. 26 is a block diagram illustrating an example of a hardware configuration that achieves a calculation device according to each example embodiment of the present invention.

### EXAMPLE EMBODIMENTS

Hereinafter, example embodiments of the present invention will be described with reference to the drawings. However, example embodiments described below have technically preferable limitations for carrying out the present invention, but the scope of the invention is not limited to the following. In all the drawings used in the following description of the example embodiment, the same reference numerals are given to the same parts unless there is a particular reason. Further, in the following example embodiments, repeated description of similar configurations and operations may be omitted. In addition, directions of arrows in the drawings illustrate an example, and do not limit directions of signals between blocks.

### First Example Embodiment

First, a gait measurement system according to a first example embodiment of the present invention will be described with reference to the drawings. The gait measurement system according to the present example embodiment calculates a symmetry of walking using sensor data acquired by a sensor disposed on footwear such as a shoe. The symmetry of walking is an index representing a symmetry of a walking state of both feet during walking. Hereinafter, an example of using sensor data acquired by the sensor disposed on the footwear such as a shoe will be described, but sensor data acquired by a sensor attached to an ankle or a foot may be used.

Hereinafter, an example will be described in which the gait measurement system calculates a walking parameter using sensor data acquired by an acceleration sensor and an angular velocity sensor disposed on the footwear, and calculates the symmetry of walking using the calculated walking parameter. The walking parameter is a parameter such as an attitude angle or a sensor height calculated using a physical quantity such as the acceleration or angular velocity.

### Configuration

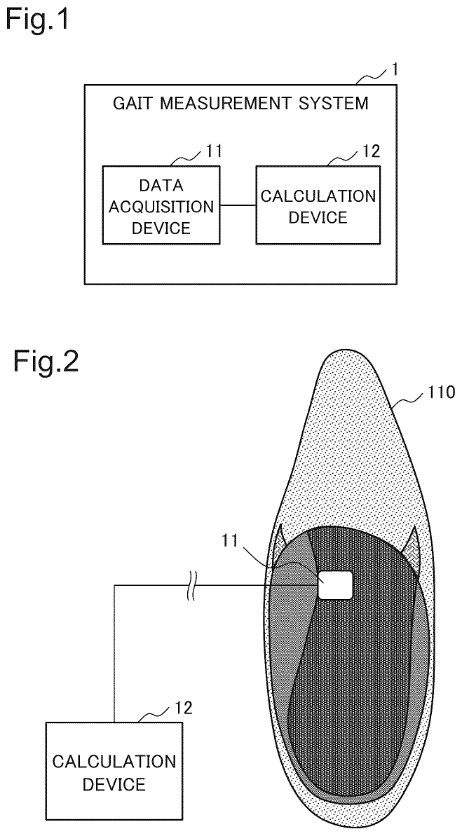

FIG. 1 is a block diagram schematically illustrating a configuration of the gait measurement system 1 according to the present example embodiment. The gait measurement system 1 includes a data acquisition device 11 and a calculation device 12. The data acquisition device 11 and the calculation device 12 may be connected by a wired or wireless manner. In addition, the data acquisition device 11 and the calculation device 12 may be configured by a single device. The data acquisition device 11 may be excluded from the configuration of the gait measurement system 1, and only the calculation device 12 may constitute the gait measurement system 1.

The data acquisition device 11 is connected to the calculation device 12. The data acquisition device 11 has at least an acceleration sensor and an angular velocity sensor. For example, the data acquisition device 11 is installed on a user's footwear. The data acquisition device 11 converts physical quantities related to movement such as acceleration or angular velocity acquired by an acceleration sensor and an angular velocity sensor into digital data (also referred to as sensor data), and transmits the converted sensor data to the calculation device 12. Note that the data acquisition device 11 may be configured to be worn on an ankle or a foot instead of a shoe.

The data acquisition device 11 is achieved by, for example, an inertial measurement unit including the acceleration sensor and the angular velocity sensor. An example of the inertial measurement device is an inertial measurement unit (IMU). The IMU includes a triaxial acceleration sensor and a triaxial angular velocity sensor. Further, an example of the inertial measurement unit may include vertical gyro (VG). The VG has the same configuration as the IMU, and can output a roll angle and a pitch angle based on a direction of gravity by a technique called strap-down. Further, an example of the inertial measurement unit may include an attitude heading reference system (AHRS). The AHRS has a configuration in which an electronic compass is added to the VG. The AHRS can output a yaw angle in addition to the roll angle and the pitch angle. Further, an example of the inertial measurement unit may include a global positioning system/inertial navigation system (GPS/INS). The GPS/INS has a configuration in which the GPS is added to the AHRS. The GPS/INS may calculate a position in 3D space in addition to the roll angle, the pitch angle, and the yaw angle, and thus, can estimate a position with high accuracy.

FIG. 2 is a conceptual diagram illustrating an example in which the data acquisition device 11 is installed in a shoe 110. In the example of FIG. 2, the data acquisition device 11 is installed at a position relevant to a back side of an arch of foot. The position where the data acquisition device 11 is installed may be a position other than the back side of the arch of the foot as long as the position is inside or on the surface of the shoe 110. For example, the data acquisition device 11 may be installed on a back side of a toe or a heel.

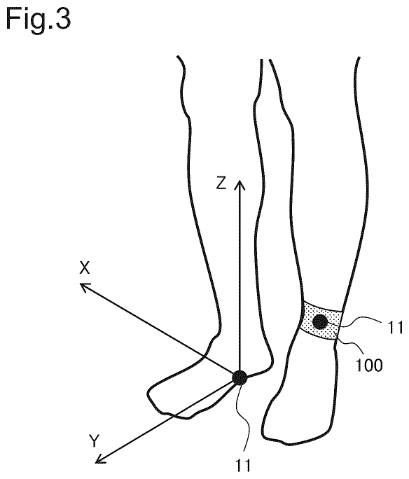

FIG. 3 is a conceptual diagram for describing coordinate systems (X-axis, Y-axis, and Z-axis) set in the data acquisition device 11 when the data acquisition device 11 is installed on a back side of an arch of a right foot. FIG. 3 illustrates an example in which a lateral direction of a pedestrian is set to an X-axis direction (rightward direction is positive), a traveling direction of the pedestrian is set to a Y-axis direction (forward direction is positive), and a direction of gravity is set to a Z-axis direction (vertically upward direction is positive). The data acquisition device 11 may be configured to be worn on an ankle or a foot. FIG. 3 illustrates an example in which the data acquisition device 11 is fixed at a position of an ankle of a left foot by a band 100. For example, the data acquisition device 11 may be fixed to a position of an ankle or a foot by a sock, a supporter, or the like. FIG. 3 illustrates that the data acquisition device 11 is installed at the back side of the arch of the right foot and the position of the ankle of the left foot, but does not illustrate that the data acquisition device 11 is installed at both the back side of the arch of the right foot and the ankle of the left foot. Usually, the data acquisition device 11 is preferably installed at the same positions of the left and right feet or ankles.

The calculation device 12 receives sensor data from the data acquisition device 11. The calculation device 12 calculates the symmetry of the walking parameters using the received sensor data. The calculation device 12 outputs the calculated symmetry of the walking parameters. For example, the calculation device 12 calculates the symmetry SIf of the walking parameters using the following Equation 1.

SIf=(F_{R}−F_{L})/(F_{R}+F_{L}) (1)

However, in the above Equation 1, each of F_{R} and F_{L} is the walking parameters of the right foot and the left foot. Examples of the walking parameter include an attitude angle and a sensor height.

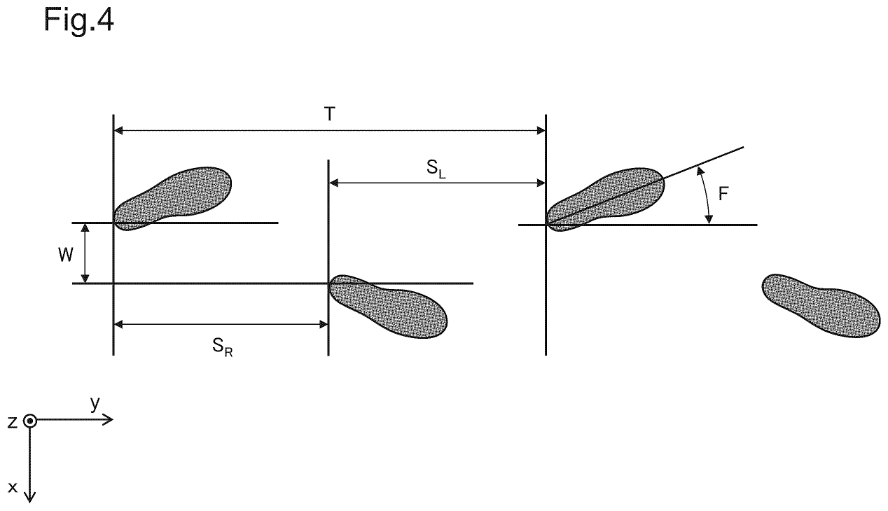

Here, the walking parameters will be described with some examples. FIGS. 4 to 6 are conceptual diagrams for describing an example of the walking parameters.

FIG. 4 illustrates a step length S_{R} of the right foot, a step length S_{L} of the left foot, a stride length T, a step width W, and a foot angle F. The step length S_{R} of the right foot is a distance of one step of the right foot. In FIG. 4, the step length S_{R} of the right foot is a difference in a Y coordinate between the heel of the right foot and the heel of the left foot when a state in which a sole of the left foot is grounded transitions to a state in which the heel of the right foot swung out in the traveling direction is landed. The step length S_{L} of the left foot is a distance relevant to one step of the left foot. In FIG. 4, the step length S_{L} of the right foot is a difference in the Y coordinate between the heel of the right foot and the heel of the left foot when a state in which a sole of the right foot is grounded transitions to a state in which the heel of the right foot swung out in the traveling direction is landed. The stride length T is a distance of two steps. The stride length T is a sum of the step length S_{R} of the right foot and the step length S_{L} of the left foot. The step width W is an interval between the right foot and the left foot. In FIG. 4, the step width W is a difference between an X coordinate of a center line of the heel of the right foot in the grounded state and an X coordinate of a center line of the heel of the left foot in the grounded state, in one step. The foot angle F is an angle formed by the center line of the foot and the traveling direction (Y axis) in the state where the back surface of the foot is grounded.

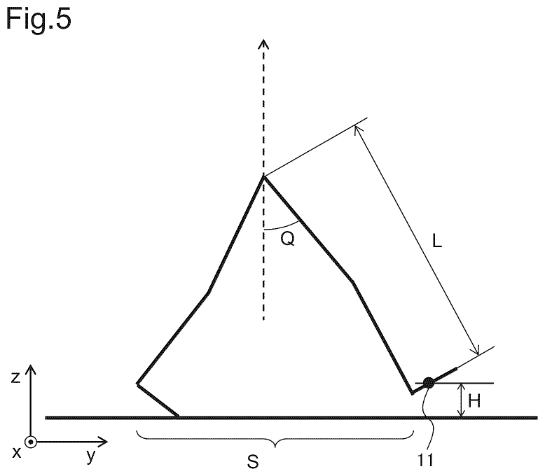

FIG. 5 illustrates a forefoot angle Q, a lower limb length L, and a sensor height H. The forefoot angle Q is also expressed as forward foot placement relative to the trunk (FFP), and is an angle formed by a central axis of a thigh of a leg swung out forward and the direction of gravity (Z axis). The lower limb length L is a length of a leg of a pedestrian. The sensor height H is a height of the data acquisition device 11 with respect to a floor plane (XY plane). Hereinafter, the floor plane is also referred to as a horizontal plane.

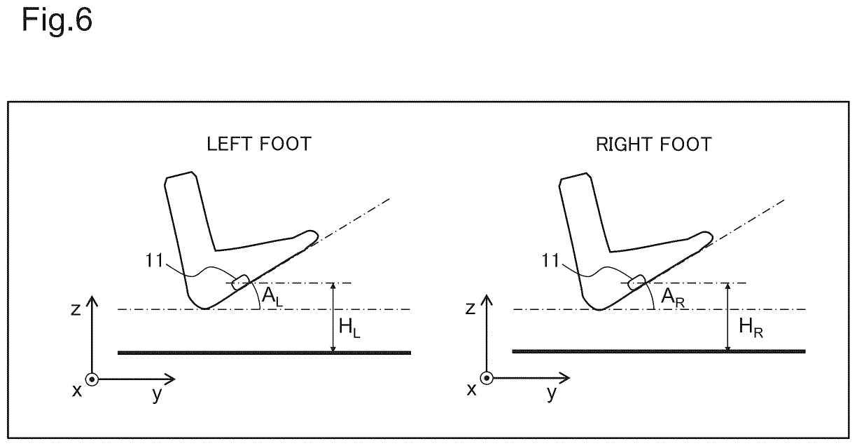

FIG. 6 illustrates a right foot sensor height H_{R}, a left foot sensor height H_{L}, a right foot attitude angle A_{R}, and a left foot attitude angle A_{L}. The right foot sensor height H_{R} is the height with respect to the horizontal plane (XY plane) of the data acquisition device 11 installed on the shoe of the right foot. The left foot sensor height H_{L} is the height with respect to the horizontal plane (XY plane) of the data acquisition device 11 installed on the shoe of the left foot. The right foot attitude angle A_{R} is an attitude angle of the right foot. The left foot attitude angle A_{L} is an attitude angle of the left foot.

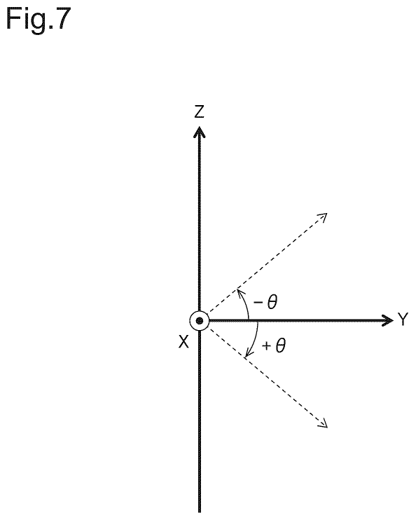

FIG. 7 is a conceptual diagram for describing the coordinate system of the attitude angle calculated by the calculation device 12. In the present example embodiment, the attitude angle indicates the angle of the back surface of the foot with respect to the horizontal plane (XY plane). In FIG. 7, the attitude angle is an angle formed by the ground (positive direction of the Y axis) and the back surface of the foot (dashed arrow). In the present example embodiment, the attitude angle associated with the upward rotation around the X axis is negative (−θ), and the attitude angle associated with the downward rotation around the X axis is positive (+θ). In other words, looking down on the ZY plane from a positive position on the X axis, a clockwise rotation around the X axis is positive (+θ), and a counterclockwise rotation is negative (−θ).

For example, the calculation device 12 calculates the attitude angle using a magnitude of the acceleration in each of the X-axis and Y-axis directions. Further, for example, the calculation device 12 can calculate the attitude angle around the X axis, the Y axis, and the Z axis by integrating the values of the angular velocities with each of the X axis, the Y axis, and the Z axis as the central axis. Acceleration data includes high frequency noise that changes in various directions, and angular velocity data always includes low frequency noise in the same direction. Therefore, when the acceleration data passes through a low-pass filter to remove a high-frequency component and the angular velocity data passes through a high-pass filter to remove a low-frequency component, the accuracy of the sensor data from the foot where noise is likely to be included can be improved. Further, the accuracy of the sensor data can be improved by applying a complementary filter to each of the acceleration data and the angular velocity data and taking a weighted average.

The calculation device 12 calculates the attitude angles of both feet using at least one of an angular velocity vector and an acceleration vector, and generates time-series data of the attitude angles of both feet. For example, the calculation device 12 generates time-series data of an attitude angle at a predetermined timing or a time interval set according to a general gait cycle or a gait cycle peculiar to a user. For example, the calculation device 12 continues to generate the time-series data of the attitude angle for a period during which user's walking is continued. The timing when the calculation device 12 generates time-series data of the attitude angle can be randomly set.

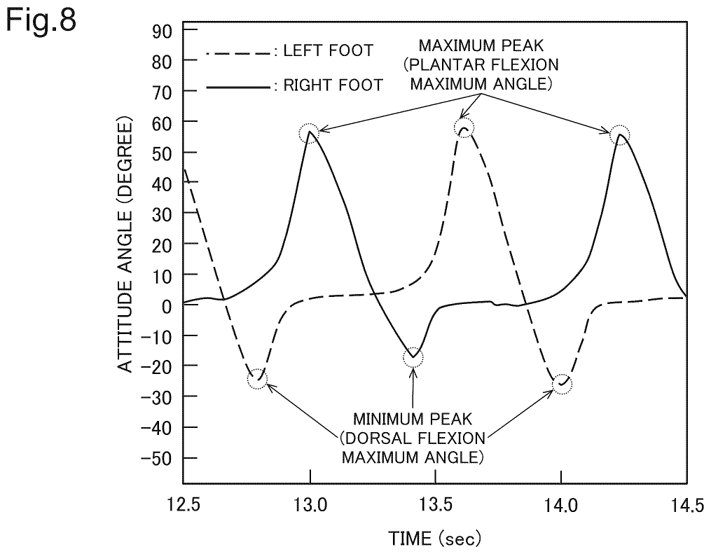

FIG. 8 is a graph illustrating an example of time-series data of an attitude angle of a pedestrian who simulatively walks with asymmetrical left and right walking. FIG. 8 illustrates an example in which the step length S_{L} of the left foot is larger than that of the step length S_{R} of the right foot. In FIG. 8, the time-series data of the attitude angle of the right foot is shown by a solid line, and the time-series data of the attitude angle of the left foot is shown by a broken line. The attitude angle turns to be negative (−θ) in the state (dorsal flexion) where the toe is above the heel and positive (+θ) in the state (plantar flexion) where the toe is below the heel. Hereinafter, the attitude angle in the state (dorsal flexion) where the toe is above the heel is referred to as a dorsal flexion angle, and the attitude angle in the state (plantar flexion) where the toe is below the heel is referred to as a plantar flexion angle.

As illustrated in FIG. 8, a maximum peak and a minimum peak appear alternately in the time-series data of the attitude angle. The minimum peak appears at the timing when the dorsal flexion angle turns to be maximum in one gait cycle. Meanwhile, the maximum peak appears at the timing when the plantar flexion angle turns to be maximum in one gait cycle. When the left and right walking is asymmetrical, the difference between the left and right is larger for the peak (minimum peak) with the maximum dorsal flexion angle than for the peak (maximum peak) with the maximum plantar flexion angle. That is, the peak (minimum peak) with the maximum dorsal flexion angle is more suitable as an index for evaluating the symmetry of walking than the peak (maximum peak) with the maximum plantar flexion angle.

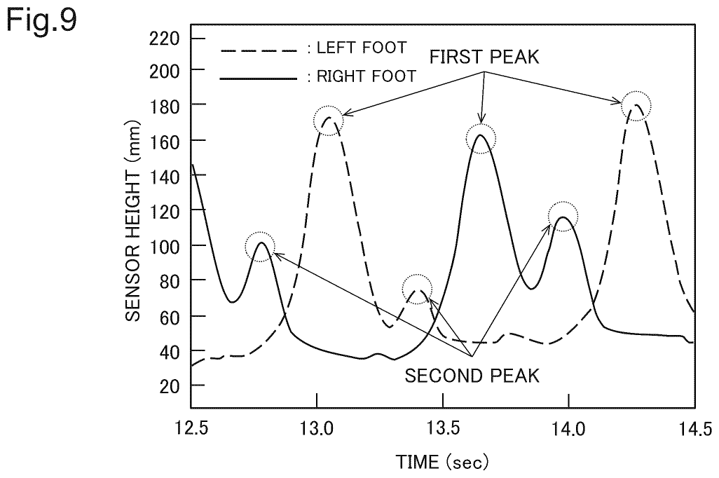

FIG. 9 is a graph illustrating an example of time-series data of a sensor height of a pedestrian who simulatively walks with the asymmetrical left and right walking. FIG. 9 illustrates an example in which the step length S_{L} of the left foot is larger than that of the step length S_{R} of the right foot. In FIG. 9, the time-series data of the sensor height of the right foot is shown by a solid line, and the time-series data of the sensor height of the left foot is shown by a broken line.

As illustrated in FIG. 9, a first maximum peak (also referred to as a first peak) and a second maximum peak (also referred to as a second peak) appear alternately in the time-series data of the sensor height. The first peak appears at the timing when the height of the foot swung out forward turns to be maximum. The second peak appears at the timing when the dorsal flexion angle turns to be maximum immediately before the heel of the foot swung out forward is landed. When the left and right walking is asymmetrical, the difference between the left and right is larger in the second peak than in the first peak. That is, the second peak is more suitable as an index for evaluating the symmetry of walking than the first peak.

In the present example embodiment, based on the measurement examples of the time-series data in FIGS. 8 and 9, an example of calculating the symmetry of the walking parameters using the attitude angle (also referred to as the maximum dorsal flexion angle) at which the dorsal flexion angle turns to be maximum in one gait cycle and the sensor height of the second peak will be described. A specific method of calculating the symmetry of the walking parameters will be described later.

The outline of the configuration of the gait measurement system 1 of the present example embodiment has been described above. The configuration of FIG. 1 is an example, and the gait measurement system 1 of the present example embodiment is not limited to the configuration of FIG. 1. For example, the gait measurement system 1 can be achieved by the IMU including the data acquisition device 11 and the calculation device 12. In addition, for example, the gait measurement system 1 can be achieved by the IMU including the data acquisition device 11, and a mobile terminal or a server including the calculation device 12.

### Data Acquisition Device

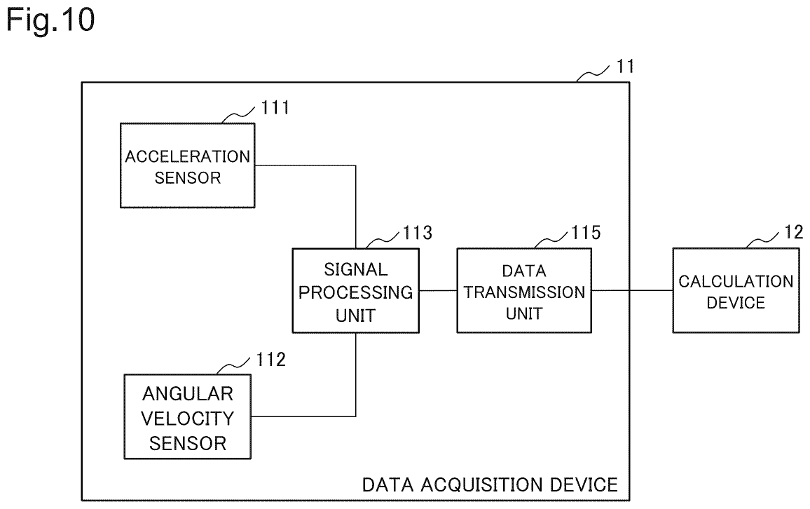

Next, details of the data acquisition device 11 included in the gait measurement system 1 will be described with reference to the drawings. FIG. 10 is a block diagram illustrating an example of a detailed configuration of the data acquisition device 11. The data acquisition device 11 includes an acceleration sensor 111, an angular velocity sensor 112, a signal processing unit 113, and a data transmission unit 115.

The acceleration sensor 111 is a sensor that measures acceleration in triaxial direction. The acceleration sensor 111 is connected to the signal processing unit 113. The acceleration sensor 111 outputs the measured acceleration to the signal processing unit 113.

The angular velocity sensor 112 is a sensor that measures angular velocity in the triaxial direction. The angular velocity sensor 112 is connected to the signal processing unit 113. The angular velocity sensor 112 outputs the measured angular velocity to the signal processing unit 113.

The signal processing unit 113 is connected to the acceleration sensor 111, the angular velocity sensor 112, and the data transmission unit 115. The signal processing unit 113 acquires acceleration and angular velocity from each of the acceleration sensor 111 and the angular velocity sensor 112. The signal processing unit 113 converts the acquired acceleration and angular velocity into digital data, and outputs the converted digital data (also referred to as sensor data) to the data transmission unit 115. The sensor data includes at least acceleration data (including acceleration vectors in triaxial direction) obtained by converting acceleration of analog data into digital data and angular velocity data (including angular velocity vectors in triaxial direction) obtained by converting angular velocity of analog data into digital data. Acquisition times of the acceleration data and the angular velocity data are associated with the acceleration data and the angular velocity data. In addition, the signal processing unit 113 may be configured to output sensor data obtained by adding correction such as a mounting error, temperature correction, and linearity correction to the acquired acceleration data and angular velocity data.

The data transmission unit 115 is connected to the signal processing unit 113. In addition, the data transmission unit 115 is connected to the calculation device 12. The data transmission unit 115 acquires sensor data from the signal processing unit 113. The data transmission unit 115 transmits the acquired sensor data to the calculation device 12. The data transmission unit 115 may transmit the sensor data to the calculation device 12 via a wire such as a cable, or may transmit the sensor data to the calculation device 12 via wireless communication. For example, the data transmission unit 115 is configured to transmit the sensor data to the calculation device 12 via a wireless communicator capability (not illustrated) conforming to a standard such as Bluetooth (registered trademark) or Wi-Fi (registered trademark). The communicator capability of the data transmission unit 115 may conform to a standard other than Bluetooth (registered trademark) or Wi-Fi (registered trademark).

The details of configuration of the data acquisition device 11 have been described above. The configuration of FIG. 10 is an example, and the configuration of the data acquisition device 11 included in the gait measurement system 1 of the present example embodiment is not limited to the form of FIG. 10.

### Calculation Device

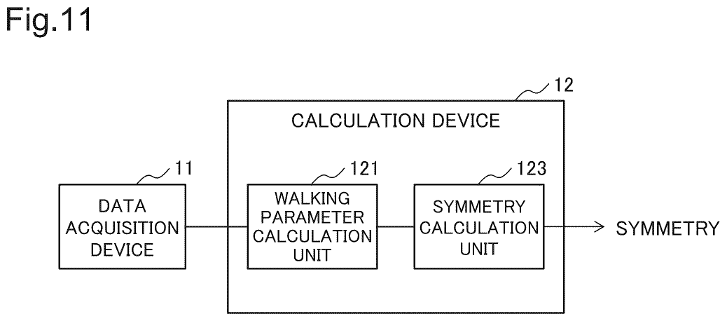

Next, details of the calculation device 12 included in the gait measurement system 1 will be described with reference to the drawings. FIG. 11 is a block diagram illustrating an example of a configuration of the calculation device 12. The calculation device 12 includes a walking parameter calculation unit 121 and a symmetry calculation unit 123.

The walking parameter calculation unit 121 is connected to the data acquisition device 11. Furthermore, the walking parameter calculation unit 121 is connected to the symmetry calculation unit 123. The walking parameter calculation unit 121 acquires at least one of acceleration data and angular velocity data for left and right feet from the data acquisition device 11. The walking parameter calculation unit 121 synchronizes data according to an acquisition time of data in the data acquisition devices 11 installed in the left and right shoes, and calculates walking parameters using the data. The walking parameter calculation unit 121 generates time-series data of the walking parameters of both feet using the calculated walking parameter. The walking parameter calculation unit 121 outputs the generated time-series data of the walking parameter of both feet to the symmetry calculation unit 123.

For example, the walking parameter calculation unit 121 calculates attitude angles of both feet using at least one of acceleration data and angular velocity data. The walking parameter calculation unit 121 generates time-series data of the attitude angles of both feet using attitude angles for several steps. The walking parameter calculation unit 121 outputs the generated time-series data of the attitude angles of both feet to the symmetry calculation unit 123.

For example, the walking parameter calculation unit 121 calculates a sensor height using the acceleration data and the angular velocity data. For example, the walking parameter calculation unit 121 sets the sensor height in a state where a foot is grounded as the initial state, and calculates a movement amount from the initial state using the acceleration data and the angular velocity data to calculate the sensor height. The walking parameter calculation unit 121 generates time-series data of the sensor heights of both feet by using the sensor height for several steps. The walking parameter calculation unit 121 outputs the generated time-series data of the sensor heights of both feet to the symmetry calculation unit 123.

For example, the walking parameter calculation unit 121 calculates the attitude angles around the X axis, the Y axis, and the Z axis by integrating the values of the angular velocities with each of the X axis, Y axis, and Z axis as the central axes. For example, the attitude angle is represented by a roll angle θᵣₒₗₗ, a pitch angle θ_{pitch}, and a yaw angle θ_{yaw}. Each of the roll angle θᵣₒₗₗ, the pitch angle θ_{pitch}, and the yaw angle θ_{yaw} represents rotation about each of the Y, X, and Z axes as a central axis.

The angular velocity data includes an error mainly caused by a bias. The error included in the angular velocity data is accumulated by the integration. Therefore, the attitude angle may be calculated using the acceleration data by the Madgwick method disclosed in NPL 1 below.

NPL 1: S. Madgwick, A. Harrison, R. Vaidyanathan, “Estimation of IMU and MARG orientation using a gradient descent algorithm,” 2011 IEEE International Conference on Rehabilitation Robotics, Rehab Week Zurich, ETH Zurich Science City, Switzerland, June 29 -July 1, pp. 179-185, 2011.

According to the technique of Madgwick disclosed in NPL 1, accumulation of errors can be reduced by integrating and using measurement data of angular velocity and measurement data of acceleration based on gravitational acceleration.

The symmetry calculation unit 123 is connected to the walking parameter calculation unit 121. In addition, the symmetry calculation unit 123 is connected to an external system or device (not illustrated). The symmetry calculation unit 123 acquires the walking parameters of both feet from the walking parameter calculation unit 121. The symmetry calculation unit 123 calculates the symmetry of the walking parameters using the walking parameters of both feet. For example, the symmetry calculation unit 223 calculates the symmetry of the attitude angles and the sensor height as the symmetry of the walking parameters. The symmetry calculation unit 223 may calculate an arithmetic mean or a geometric mean of the symmetry of the attitude angles and the symmetry of the sensor heights as the symmetry of the walking parameters. The symmetry calculation unit 123 outputs information on the calculated symmetry to the external system or device (not illustrated).

For example, in a case where the attitude angle is used as the walking parameter, the symmetry calculation unit 123 acquires time-series data of the attitude angles of both feet from the walking parameter calculation unit 121. The symmetry calculation unit 123 detects an attitude angle (referred to as a dorsal flexion maximum angle) indicating a minimum peak from the time-series data of each attitude angles of both feet. The symmetry calculation unit 123 calculates symmetry SIa of the attitude angle using the detected dorsal flexion maximum angle. For example, the calculation device 12 calculates the symmetry SIa of the attitude angle using the following Equation 2.

SIa=(A_{R}−A_{L})/(A_{R}+A_{L}) (2)

However, in the above Equation 2, each of A_{R} and A_{L} is the dorsal flexion maximum angles of the right foot and the left foot. The equation for calculating the symmetry SIa of the attitude angle is not limited to the above Equation 2.

For example, in a case where the sensor height is used as the walking parameter, the symmetry calculation unit 123 acquires the time-series data of the sensor heights of both feet from the walking parameter calculation unit 121. The symmetry calculation unit 123 detects a maximum peak from the time-series data of the sensor heights of both feet. From the time-series data of the sensor height for one step, a relatively large maximum peak (first peak) and a relatively small maximum peak (second peak) following the first peak are detected. The symmetry calculation unit 123 calculates symmetry SIh of the sensor height using the second peak. For example, the calculation device 12 calculates the symmetry SIh of the sensor height by using the following Equation 3.

SIh=(H_{R}−H_{L})/(H_{R}+H_{L}) (3)

However, in Equation 3 above, each of H_{R} and H_{L} is the sensor heights at the second peaks of the right foot and the left foot.

In addition, for example, the symmetry calculation unit 123 may calculate the symmetry SIh of the sensor heights using both the first peak and the second peak. For example, the calculation device 12 calculates the symmetry SIh of the sensor heights by using the following Equation 4 or 5.

SIh=H_{R}/P_{R}−H_{L}/P_{L} (4)

SIh=H_{R}/P_{R}+H_{L}/P_{L} (5)

However, in Equations 4 and 5 above, each of P_{R} and P_{L} is the sensor heights at the first peaks of the right foot and the left foot. The equation for calculating the symmetry SIh of the sensor heights is not limited to the above Equations 3 to 5.

The details of the configuration of the calculation device 12 have been described above. The configuration of FIG. 11 is an example, and the configuration of the calculation device 12 included in the gait measurement system 1 of the present example embodiment is not limited to the form of FIG. 11.

The walking parameter calculation unit 121 and the symmetry calculation unit 123 constituting the calculation device 12 may be distributed to different devices. For example, the walking parameter calculation unit 121 may be included in the IMU, and the symmetry calculation unit 123 may be included in the mobile terminal or the server.

### Operation

Next, an example of an operation of the calculation device 12 of the present example embodiment will be described with reference to the drawings. Hereinafter, each operation of the walking parameter calculation unit 121 and the symmetry calculation unit 123 included in the calculation device 12 will be individually described.

### Walking Parameter Calculation Unit

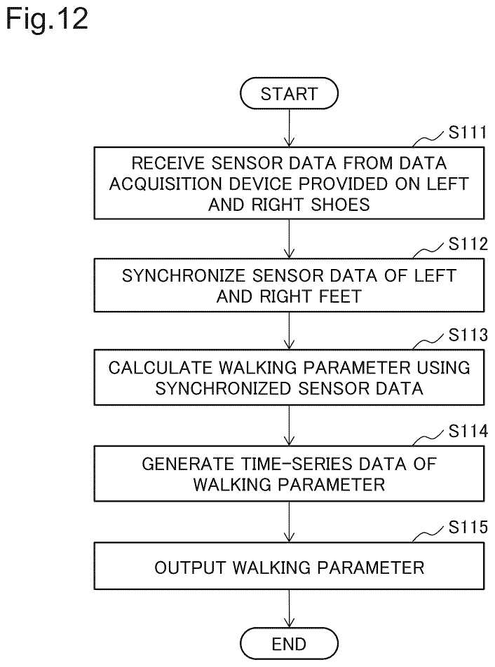

FIG. 12 is a flowchart for describing an example of the operation of the walking parameter calculation unit 121 of the calculation device 12. In the following description along the flowchart of FIG. 12, the walking parameter calculation unit 121 is an operation subject.

In FIG. 12, first, the walking parameter calculation unit 121 acquires the sensor data of the left and right feet from each of the data acquisition devices 11 installed in left and right shoes (step S111).

Next, the walking parameter calculation unit 121 calculates the sensor data of the left and right feet (step S112)

Next, the walking parameter calculation unit 121 calculates the walking parameters of the left and right feet by using at least one of the acceleration data and the angular velocity data included in the sensor data of the left and right feet (step S113). For example, the calculation device 12 calculates the walking parameters such as the attitude angle or the sensor height.

Next, the walking parameter calculation unit 121 generates the time-series data of the walking parameters of the left and right feet (step S114).

Then, the walking parameter calculation unit 121 outputs the generated time-series data of the walking parameters of the left and right feet to the symmetry calculation unit 123 (step S115).

### Symmetry Calculation Unit

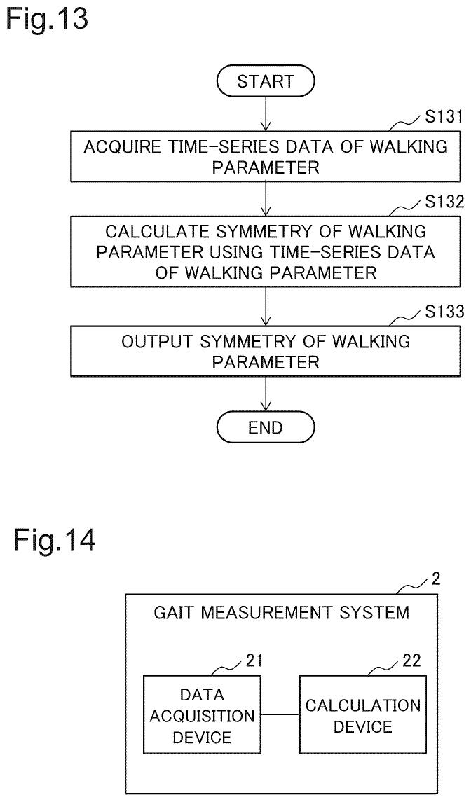

FIG. 13 is a flowchart for describing an example of an operation of the symmetry calculation unit 123 of the calculation device 12. In the following description along the flowchart of FIG. 13, the symmetry calculation unit 123 is an operation subject.

In FIG. 13, first, the symmetry calculation unit 123 acquires the time-series data of the walking parameters of the left and right feet from the walking parameter calculation unit 121 (step S131).

Next, the symmetry calculation unit 123 calculates the symmetry of the walking parameters by using the acquired time-series data of the walking parameters of the left and right feet (step S132). For example, the calculation device 12 calculates the symmetry of the walking parameters using the time-series data of the walking parameter such as the attitude angle or the sensor height.

The symmetry calculation unit 123 outputs the calculated symmetry of the walking parameters (step S133).

An example of the operation of the calculation device 12 of the present example embodiment has been described above. The flowcharts of FIGS. 12 and 13 are examples, and the operation of the calculation device 12 of the present example embodiment is not limited to the processing along the flowcharts of FIGS. 12 and 13.

As described above, the gait measurement system of the present example embodiment includes a data acquisition device configured to measure physical quantities related to movements of both left and right feet, and a calculation device configured to calculate a symmetry of walking using the physical quantities related to the movements of the left and right feet. According to the present example embodiment, it is possible to easily measure the symmetry of walking in day-to-day life.

The gait measurement system according to one aspect of the present example embodiment includes a walking parameter calculation unit and a symmetry calculation unit. The walking parameter calculation unit generates time-series data of the walking parameter using the physical quantities related to the movements of the left and right feet. The symmetry calculation unit calculates the symmetry of the walking parameters of the left and right feet as the symmetry of walking using the time-series data of the walking parameters of the left and right feet.

Further, in one aspect of the present example embodiment, the data acquisition device measures at least one of the acceleration in the triaxial direction and the angular velocity in the triaxial direction as the physical quantity. The walking parameter calculation unit generates the time-series data of the attitude angles of the left and right feet by using at least one of the acceleration in the triaxial direction and the angular velocity in the triaxial direction measured by the data acquisition device. The symmetry calculation unit calculates the symmetry of the walking parameters by using maximum values of peaks appearing in the time-series data of the attitude angles of the left and right feet. For example, the symmetry calculation unit calculates the symmetry of the walking parameters by using a maximum value at a time when the dorsal flexion angle turns to be maximum among the maximum values of the peaks appearing in the time-series data of the attitude angles of the left and right feet.

Further, in one aspect of the present example embodiment, the data acquisition device measures at least one of the acceleration in the triaxial direction and the angular velocity in the triaxial direction as the physical quantity. The walking parameter calculation unit generates the time-series data of the sensor heights of the left and right feet by using at least one of the acceleration in the triaxial direction and the angular velocity in the triaxial direction measured by the data acquisition device. The symmetry calculation unit calculates the symmetry of the walking parameters by using maximum values of peaks appearing in the time-series data of the sensor heights of the left and right feet. For example, the symmetry calculation unit calculates the symmetry of the walking parameters by using a maximum value at a time when a dorsal flexion angle turns to be maximum immediately before a heel of foot swung out forward is landed, among the maximum values of the peaks appearing in the time-series data of the sensor heights of the left and right feet.

According to one aspect of the present example embodiment, it is possible to accurately measure the symmetry of walking using the physical quantity related to the movement measured by the data acquisition device installed on the footwear such as the shoe without using a large-scale device. That is, according to one aspect of the present example embodiment, it is possible to accurately measure the symmetry of walking in day-to-day life.

### Second Example embodiment

Next, a gait measurement system according to a second example embodiment of the present invention will be described with reference to the drawings. The gait measurement system of the present example embodiment is different from the gait measurement system of the first example embodiment in that step lengths are calculated from a symmetry of walking parameters by applying a regression model that associates the symmetry of the walking parameters with a symmetry of step lengths. Hereinafter, the description of the same configuration or operation as those of the first example embodiment may be omitted.

### Configuration

FIG. 14 is a block diagram schematically illustrating a configuration of a gait measurement system 2 according to the present example embodiment. The gait measurement system 2 includes a data acquisition device 21 and a calculation device 22. The data acquisition device 21 and the calculation device 22 may be connected by a wired or wireless manner. In addition, the data acquisition device 21 and the calculation device 22 may be configured by a single device. The data acquisition device 21 may be excluded from the configuration of the gait measurement system 2, and only the calculation device 22 may constitute the gait measurement system 2.

The data acquisition device 21 is connected to the calculation device 22. The data acquisition device 21 has at least an acceleration sensor and an angular velocity sensor. The data acquisition device 21 converts data acquired by the acceleration sensor and the angular velocity sensor into digital data. The data acquisition device 21 transmits sensor data including an acceleration vector and an angular velocity vector after conversion into the digital data to the calculation device 22. The data acquisition device 21 has a configuration relevant to the data acquisition device 11 of the first example embodiment.

The calculation device 22 is connected to the data acquisition device 21. The calculation device 22 receives sensor data from the data acquisition device 21. The calculation device 22 calculates the symmetry of the walking parameters of both feet using the received sensor data. The calculation device 22 calculates the symmetry of the step lengths of both feet from the calculated symmetry of the walking parameters of both feet using the regression model that associates the symmetry of the walking parameters with the symmetry of the step lengths. Further, the calculation device 22 calculates the step lengths of both feet using the calculated symmetry of the step lengths of both feet. The calculation device 22 outputs the calculated step lengths of both feet to an external system or device (not illustrated).

For example, the calculation device 22 uses a general-purpose regression model generated using data of a plurality of subjects. For example, the calculation device 22 uses a regression model generated using data of a plurality of subjects having a similar walking tendency (disease or injury, nature, etc.). For example, the calculation device 22 uses a regression model that is generated individually.

The outline of the configuration of the gait measurement system 2 of the present example embodiment has been described above. The configuration of FIG. 14 is an example, and the gait measurement system 2 of the present example embodiment is not limited to the configuration of FIG. 14. For example, the gait measurement system 2 can be achieved by the IMU including the data acquisition device 21 and the calculation device 22. In addition, for example, the gait measurement system 2 can be achieved by the IMU including the data acquisition device 21, and a mobile terminal or a server including the calculation device 22.

### Calculation Device

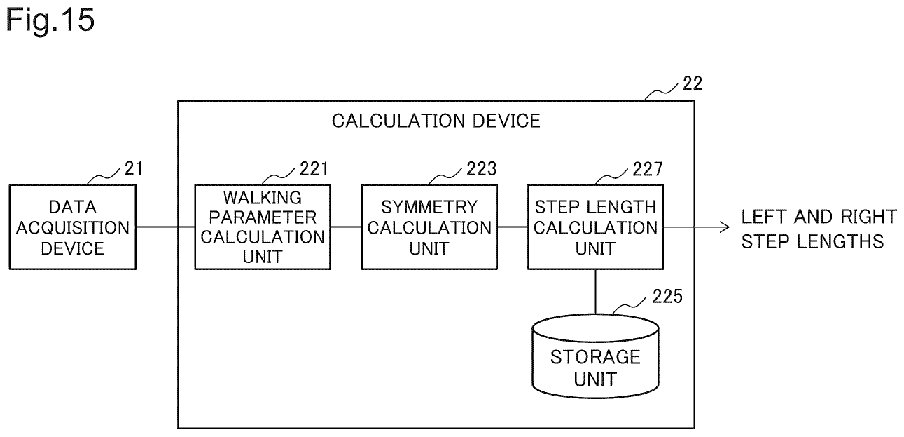

Next, details of the calculation device 22 included in the gait measurement system 2 will be described with reference to the drawings. FIG. 15 is a block diagram illustrating an example of a configuration of the calculation device 22. The calculation device 22 includes a walking parameter calculation unit 221, a symmetry calculation unit 223, a storage unit 225, and a step length calculation unit 227.

The walking parameter calculation unit 221 is connected to the data acquisition device 21. Furthermore, the walking parameter calculation unit 221 is connected to the symmetry calculation unit 223. The walking parameter calculation unit 221 acquires at least one of acceleration data and angular velocity data for the left and right feet from the data acquisition device 21. The walking parameter calculation unit 221 synchronizes the acquired data between the left and right feet, and calculates the walking parameter using the data. The walking parameter calculation unit 221 generates time-series data of the walking parameters of both feet using the calculated walking parameter. The walking parameter calculation unit 221 outputs the generated time-series data of the walking parameter of both feet to the symmetry calculation unit 223. The walking parameter calculation unit 221 has a configuration relevant to the walking parameter calculation unit 121 of the first example embodiment.

The symmetry calculation unit 223 is connected to the walking parameter calculation unit 221 and the step length calculation unit 227. The symmetry calculation unit 223 acquires the walking parameters of both feet from the walking parameter calculation unit 221. The symmetry calculation unit 223 calculates the symmetry of the walking parameters using the walking parameters of both feet. For example, the symmetry calculation unit 223 calculates the symmetry of the attitude angles and the sensor height as the symmetry of the walking parameters. The symmetry calculation unit 223 may calculate an arithmetic mean or a geometric mean of the symmetry of the attitude angles and the symmetry of the sensor heights as the symmetry of the walking parameters. The symmetry calculation unit 223 outputs the calculated symmetry of the walking parameters to the step length calculation unit 227. The symmetry calculation unit 223 has a configuration relevant to the symmetry calculation unit 123 of the first example embodiment.

The storage unit 225 is connected to the step length calculation unit 227. The storage unit 225 stores the regression model that associates the symmetry of the walking parameters with the symmetry of the step lengths. The regression model may be a universal model registered in advance in the gait measurement system 2, or may be individual models for each pedestrian.

The step length calculation unit 227 is connected to the symmetry calculation unit 223 and the storage unit 225. In addition, the step length calculation unit 227 is connected to an external system or device (not illustrated). The step length calculation unit 227 acquires the symmetry of the walking parameters from the symmetry calculation unit 223. The step length calculation unit 227 calculates the symmetry of the step lengths by applying the acquired symmetry of the walking parameters to the regression model stored in the storage unit 225. The step length calculation unit 227 calculates each of the step length of the right foot and the step length of the left foot using the calculated symmetry of the step lengths. The step length calculation unit 227 outputs each of the calculated step lengths of left and right feet.

The details of the calculation device 22 included in the gait measurement system 2 have been described above. The configuration of FIG. 15 is an example, and the calculation device 22 is not limited to the configuration of FIG. 15. For example, the walking parameter calculation unit 221, the symmetry calculation unit 223, the storage unit 225, and the step length calculation unit 227 constituting the calculation device 22 may be distributed to different devices. For example, the walking parameter calculation unit 221 may be included in the IMU, and the symmetry calculation unit 223, the storage unit 225, and the step length calculation unit 227 may be included in the mobile terminal or the server. In addition, for example, the walking parameter calculation unit 221 may be included in the IMU, and at least any one of the symmetry calculation unit 223, the storage unit 225, and the step length calculation unit 227 may be included in the mobile terminal or the server. In addition, the storage unit 225 may be stored in a storage accessible from the step length calculation unit 227 included in the mobile terminal or the server.

### Regression Model

Next, an example of generating the regression model using the relationship between the symmetry of the walking parameters such as the attitude angle and the sensor height and the symmetry of the step lengths will be described.

Hereinafter, an example of generating the regression model based on the data disclosed in NPL 2 will be described.

NPL 2: Y. Morio, et al, “The Relationship between Walking Speed and Step Length in Older Aged Patients,” Diseases, 2019 March; 7(1):17.

FIG. 2 of NPL 2 discloses an example showing that a maximum value of a walking speed and a ratio of a step length to a height of foot have a proportional relationship regardless of individual differences.

Here, a hypothesis is made that a step length S can be linearly regressed by the relationship of the following Equation 6 using a walking parameter F as a variable and a universal regression model f(F) that does not depend on individual differences.

S=C×f(F) (6)

However, in Equation 6, C denotes a coefficient.

The regression model f(F) is a model generated using the relationship between the symmetry of the walking parameters F regarding movement such as an attitude angle A and a sensor height H and the symmetry of the step lengths. The coefficient C has individual differences depending on a lower limb length L and a walking speed v. In the present example embodiment, the calculation expression of Equation 6 is compared with a calculation expression for calculating a step length S by another approach, and a parameter not depending on individual differences included in a calculation expression of another approach is set as the regression model f(F).

Assuming that a height of foot of a pedestrian depends on a lower limb length L of a pedestrian, it is estimated that there is a relationship (proportional relationship) represented by the following Equation 7 between a ratio S/L of the step length S to the lower limb length L and the walking speed v based on NPL 2.

S/L=k×v (7)

However, in Equation 7, k is a proportional constant.

Here, the relationship of the following Equation 8 is derived based on Equations 6 and 7.

C×f(F)=k×v×L (8)

In the right side of Equation 8, the walking speed v and the lower limb length L depend on individual differences, and the proportional constant k does not depend on individual differences. That is, the coefficient C is relevant to a product of the walking speed v and the lower limb length L depending on individual differences, and the regression model f(F) is relevant to a proportional coefficient k not depending on individual differences.

In general, a symmetry SIs of the step length S is calculated by the following Equation 9.

SIs=(S_{R}−S_{L})/(S_{R}+S_{L}) (9)

However, in the above Equation 9, each of S_{R} and S_{L} is the step lengths of the right foot and the left foot.

The step length (S_{R} and S_{L}) of the right and left foot in the above Equation 9 includes the walking speed v and the lower limb length L depending on individual differences. Therefore, in the present example embodiment, the symmetry SIs of the step length S is calculated using a model that does not depend on individual differences. Specifically, as described later, the symmetry SIs of the step length S is calculated using the regression model f(A) related to the attitude angle A or the regression model f(H) related to the sensor height H (see Equations 10 to 14 described later).

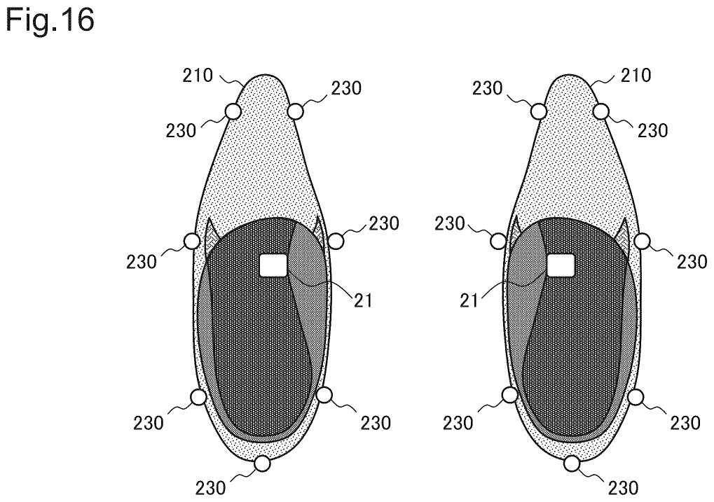

Here, an example of a specific method of generating a regression model will be described with reference to FIGS. 16 to 19. In the examples of FIGS. 16 to 19, marks for motion capture are attached to shoes, and a trajectory of foot of a pedestrian who walks while wearing the shoes is captured by a camera to generate the regression model.

FIG. 16 illustrates an example in which a plurality of marks 230 for motion capture is attached to the shoes 210 of both feet. In the example of FIG. 16, a total of 7 marks 230, 3 marks on each of the left and right side surfaces and 1 mark on the heel side surface, are attached to each of the shoes 210 of both feet. Attachment positions of the plurality of marks 230 illustrated in FIG. 16 are examples, and the attachment positions of the plurality of marks 230 are not limited to the positions illustrated in FIG. 16. Further, FIG. 16 illustrates an example in which the data acquisition device 21 is installed at a position relevant to the back side of the arch of the foot, but the data acquisition device 21 may not be installed in the shoe 210 at the time of the motion capture.

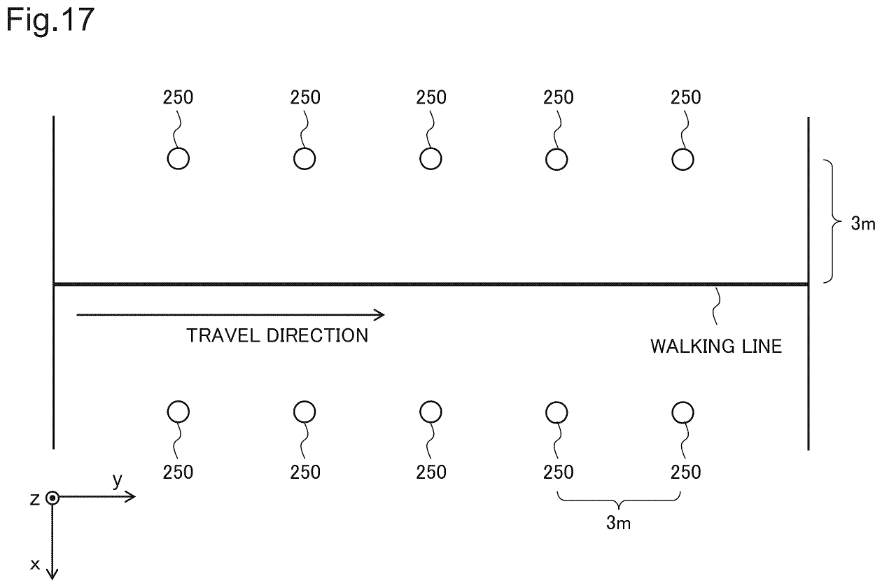

FIG. 17 is a conceptual diagram illustrating an example of a walking line when motion capturing walking of a pedestrian wearing the shoes 210 to which the plurality of marks 230 are attached, and arrangement positions of a plurality of cameras 250. In the example of FIG. 17, five cameras 250 (10 cameras in total) are arranged on each side across the walking line. Each of the plurality of cameras 250 is arranged at a height of 2 m from a horizontal plane (XY plane) and at a position of 3 m from the walking line at intervals of 3 m, focusing on the walking line on which the pedestrian walks.

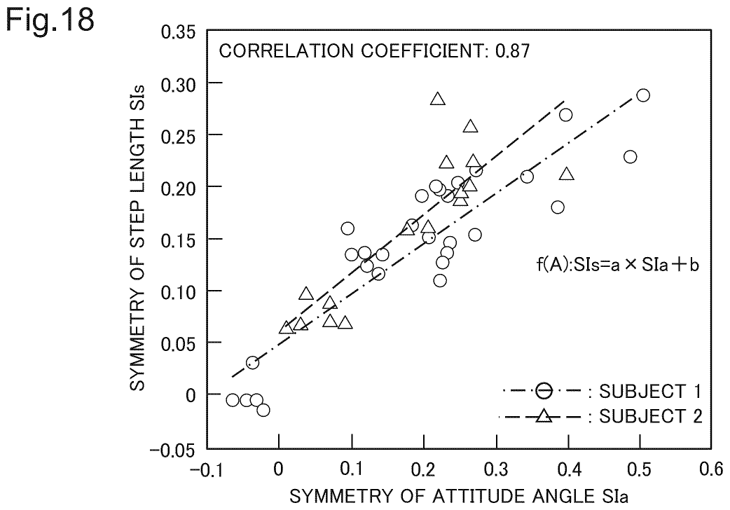

The movements of the plurality of marks 230 installed on the shoes 210 of the pedestrian walking along the walking line are analyzed using moving images captured by the plurality of cameras 250. By considering the plurality of marks 230 as one rigid body and analyzing the movement of the center of gravity thereof, it is possible to generate a regression model that associates the symmetry of the walking parameters such as the attitude angle or the sensor height with the symmetry of the step lengths. FIG. 18 illustrates an example of the relationship between the symmetry SIa of the attitude angle and the symmetry SIs of the step length obtained by motion capturing the walking of two subjects (subject 1, subject 2).

For the subject 1, a linear regression of a plot (◯) of the symmetry SIa of the attitude angle and the symmetry SIs of the step length was found to be linear (dashed-dotted line). In addition, also for the subject 2, linearity (broken line) was observed when a plot (Δ) of the symmetry SIa of the attitude angle and the symmetry SIs of the step length was linearly regressed. That is, the regression model indicating the relationship between the symmetry SIa of the attitude angle and the symmetry SIs of the step length can be individually generated for each pedestrian. When such a regression model is used, the regression models for each pedestrian may be stored in advance in the storage unit 225.

In addition, for the two subjects (subject 1, subject 2), the correlation coefficient was 0.87 when the plots (◯ and Δ) of the symmetry SIa of the attitude angles and the symmetry SIs of the step lengths were linearly regressed. That is, the regression model indicating the relationship between the symmetry SIa of the attitude angle and the symmetry SIs of the step length can be used as a universal model having generality regardless of the subject. When such a regression model is used, the existing regression model may be stored in advance in the storage unit 225 regardless of the pedestrian. For example, the regression model f(A) of the following Equation 10 in which a relational expression between the symmetry SIa of the attitude angle and the symmetry SIs of the step length obtained from the walking of the plurality of subjects is collected may be stored in the storage unit 225 in advance.

f(A):SIs=a×SIa+b (10)

In Equation 10, a denotes a proportional constant, and b denotes an intercept.

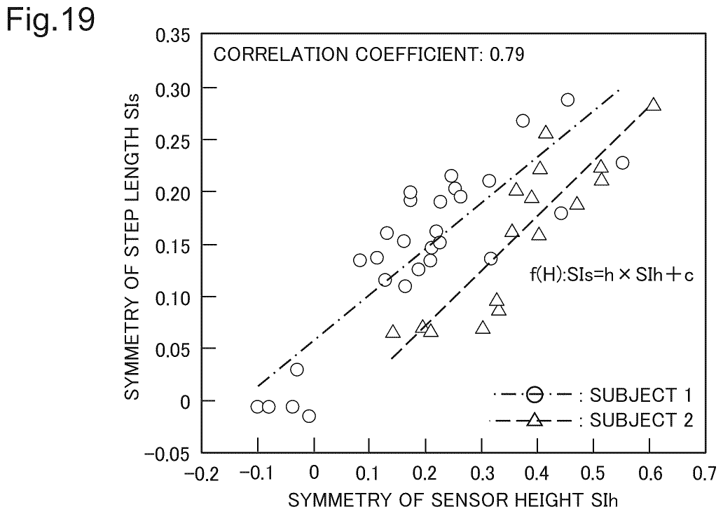

FIG. 19 illustrates the relationship between the symmetry SIh of the sensor heights and the symmetry SIs of the step lengths obtained by motion-capturing the walking of the two subjects (subject 1, subject 2).

For the subject 1, a linear regression of a plot (◯) of the symmetry SIh of the sensor heights and the symmetry SIs of the step lengths was found to be linear (dashed-dotted line). In addition, also for the subject 2, linearity (broken line) was observed when a plot (Δ) of the symmetry SIh of the sensor heights and the symmetry SIs of the step lengths was linearly regressed. That is, the regression model indicating the relationship between the symmetry SIh of the sensor heights and the symmetry SIs of the step lengths can be generated for each pedestrian. When such a regression model is used, the regression models generated for each pedestrian may be stored in advance in the storage unit 225.

In addition, for the two subjects (subject 1, subject 2), the correlation coefficient was 0.79 when the plots (◯ and Δ) of the symmetry SIh of the sensor heights and the symmetry SIs of the step lengths were linearly regressed. This indicates the possibility that the regression model indicating the relationship between the symmetry SIh of the sensor heights and the symmetry SIs of the step lengths can be used as a universal model regardless of the subject. When such a regression model is used, the existing regression model may be stored in advance in the storage unit 225 regardless of the pedestrian. For example, the regression model f(H) of the following Equation 11 in which the relational expression between the symmetry SIh of the sensor heights and the symmetry SIs of the step length obtained from the walking of the plurality of subjects is collected may be stored in the storage unit 225 in advance.

f(H):SIs=h×SIh+c (11)

In Equation 11, h denotes a proportional integer, and c denotes an intercept.

Since the sum of the step length S_{R} of the right foot and the step length S_{L} of the left foot is relevant to a stride length T (Equation 12), the difference between the step length S_{R} of the right foot and the step length S_{L} of the left foot can be expressed as the following Equation 13.

S_{R}+S_{L}=T (12)

S_{R}−S_{L}=T×SIs (13)

That is, each of the step length S_{R} of the right foot and the step length S_{L} of the left foot is put together in the following relational expression 14.

$$\{ \begin{matrix}{S}_{R} = \frac{T ( SIs + 1 )}{2} \\ {S}_{L} = \frac{T ( 1 - SIs )}{2}\end{matrix} \qquad ( 14 )$$

Hereinafter, the above Equation 14 is referred to as a relational expression U.

The step length calculation unit 227 calculates the stride length T by performing second-order integration on the acceleration measured by the data acquisition device 21 installed in the shoe of one of the left and right feet. In addition, the step length calculation unit 227 calculates the symmetry SIs of the step lengths S by applying the symmetry of the attitude angles or the sensor heights calculated from the sensor data measured by the data acquisition device 21 to the regression model. The step length calculation unit 227 calculates each of the step length S_{R} of the right foot and the step length S_{L} of the left foot by substituting the symmetry SIs of the step length S and the stride length T into the relational expression U (Equation 14).

An example of generating the regression model using the relationship between the symmetry of the walking parameters such as the attitude angle or the sensor height and the symmetry of the step length has been described above. The method of generating the regression model is an example, and the method of generating the regression model used by the gait measurement system 2 of the present example embodiment is not limited.

### Operation

Next, an example of an operation of the calculation device 22 of the present example embodiment will be described with reference to the drawings. Hereinafter, since the operations of each of the walking parameter calculation unit 221 and the symmetry calculation unit 223 included in the calculation device 22 is similar to which of the first example embodiment, only the operation of the step length calculation unit 227 will be described.

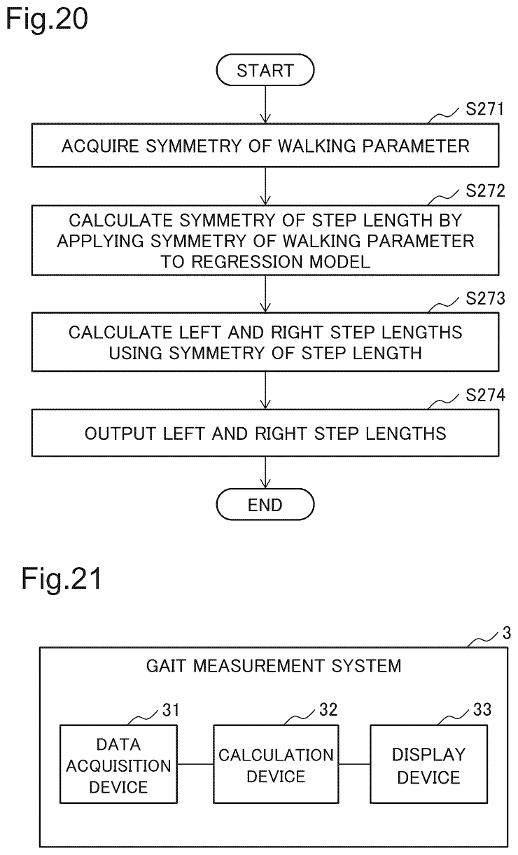

FIG. 20 is a flowchart for describing an example of the operation of the step length calculation unit 227. In the following description along the flowchart of FIG. 20, the step length calculation unit 227 is an operation subject.

In FIG. 20, first, the step length calculation unit 227 acquires the symmetry of the walking parameters from the symmetry calculation unit 223 (step S271).

Next, the step length calculation unit 227 calculates the symmetry of the step lengths by applying the symmetry of the walking parameters to the regression model (step S272).

Next, the step length calculation unit 227 calculates the step lengths of the left and right feet using the calculated symmetry of the step lengths (step S273).

Then, the step length calculation unit 227 outputs the calculated step lengths of the left and right feet (step S274).

An example of the operation of the step length calculation unit 227 of the calculation device 22 of the present example embodiment has been described above. The flowchart of FIG. 20 is an example, and the operation of the step length calculation unit 227 of the present example embodiment is not limited to the processing along the flowchart of FIG. 20.

As described above, the gait measurement system of the present example embodiment includes the calculation device including the storage unit and the step length calculation unit in addition to the walking parameter calculation unit and the symmetry calculation unit. The storage unit stores the regression model that associates the symmetry of the walking parameters with the symmetry of the step lengths. The step length calculation unit calculates the symmetry of the step lengths from the symmetry of the walking parameters using the regression model, and calculates the step lengths of the left and right feet using the calculated symmetry of the step length.

According to the present example embodiment, it is possible to accurately measure the step lengths of the left and right feet by using the physical quantity related to the movement measured by the data acquisition device installed on the footwear such as the shoe without using a large-scale device. That is, according to one aspect of the present example embodiment, it is possible to accurately measure the step lengths of the left and right feet in day-to-day life. In addition, in the present example embodiment, by using the regression model having the generality of the symmetry of walking, it is also possible to reduce the time and effort to generate the regression model again at the time of using the system.

### Third Example Embodiment

Next, a gait measurement system according to a third example embodiment of the present invention will be described with reference to the drawings. A gait measurement system of the present example embodiment is different from the gait measurement systems of the first and second example embodiments in that the gait measurement system includes a display device that displays information on a symmetry of walking. Hereinafter, the configuration in which the display device is added to the gait measurement system of the second example embodiment will be exemplified, and description of the same configuration and operation as those of the second example embodiment may be omitted.

### Configuration

FIG. 21 is a block diagram schematically illustrating a configuration of a gait measurement system 3 according to the present example embodiment. The gait measurement system 3 includes a data acquisition device 31, a calculation device 32, and a display device 33. The data acquisition device 31, the calculation device 32, and the display device 33 may be connected by a wired or wireless manner. In addition, the data acquisition device 31, the calculation device 32, and the display device 33 may be configured by a single device.

The data acquisition device 31 is connected to the calculation device 32. The data acquisition device 31 has at least an acceleration sensor and an angular velocity sensor. The data acquisition device 31 converts data acquired by the acceleration sensor and the angular velocity sensor into digital data. The data acquisition device 31 transmits sensor data including an acceleration vector and an angular velocity vector after conversion into the digital data to the calculation device 32. The data acquisition device 31 has a configuration relevant to the data acquisition device 21 of the second example embodiment.

The calculation device 32 is connected to the data acquisition device 31 and the display device 33. The calculation device 32 receives sensor data from the data acquisition device 31. The calculation device 32 calculates the symmetry of the walking parameters of both feet using the received sensor data. The calculation device 32 calculates the symmetry of the step lengths of both feet from the calculated symmetry of the walking parameters of both feet using the regression model that associates the symmetry of the walking parameters with the symmetry of the step lengths. Further, the calculation device 32 calculates the step lengths of both feet using the calculated symmetry of the step lengths of both feet. The calculation device 32 outputs information on the calculated step lengths of the left and right feet or the symmetry of the step lengths to the display device 33.

The display device 33 is connected to the calculation device 32. The display device 33 acquires information on the step lengths of the left and right feet or the symmetry of the step lengths from the calculation device 32. The display device 33 causes the display unit of the display device 33 to display information on the acquired step lengths of the left and right feet or the symmetry of the step lengths.

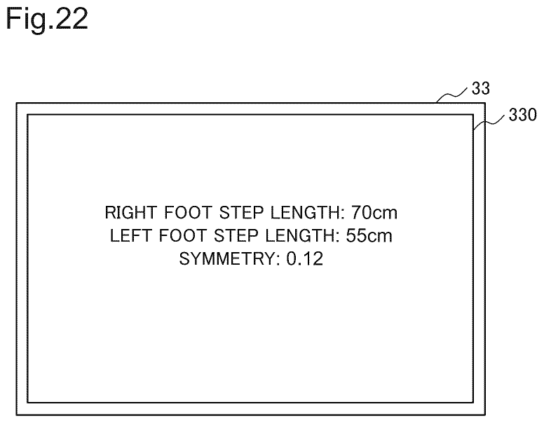

FIG. 22 illustrates an example in which the information on the step lengths of the left and right feet or the symmetry of the step lengths is displayed on the display unit 330 of the display device 33. The example of FIG. 22 is an example in which information indicating that the step length of the right foot is 70 cm, the step length of the right foot is 55 cm, and the symmetry of the left and right feet is 0.12 is displayed on the display unit 330 of the display device 33.

As illustrated in FIG. 22, a user who visually recognizes the information displayed on the display unit 330 of the display device 33 can estimate a walking state of a pedestrian according to the information displayed on the display unit 330. The information displayed on the display unit 330 is not limited to the example of FIG. 22 as long as the information is relevant to the step lengths of the left and right feet and the symmetry of the step lengths.

The outline of the configuration of the gait measurement system 3 of the present example embodiment has been described above. The configuration of FIG. 21 is an example, and the gait measurement system 3 of the present example embodiment is not limited to the configuration of FIG. 21. For example, the gait measurement system 3 can be achieved by an IMU including the data acquisition device 31 and the calculation device 32, and a mobile terminal or a computer including the display device 33. In addition, for example, the gait measurement system 3 can be achieved by the IMU including the data acquisition device 31, and the mobile terminal or the computer including the calculation device 32 and the display device 33. In addition, for example, the gait measurement system 3 can be achieved by an IMU including the data acquisition device 31, a server including the calculation device 32, and the mobile terminal or the computer including the display device 33.

### Operation

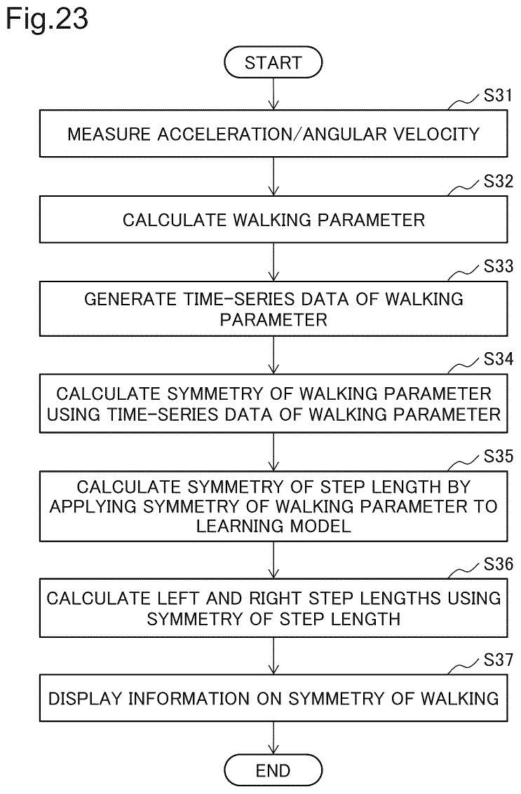

Next, an operation of the gait measurement system 3 according to the present example embodiment will be described with reference to the drawings. FIG. 23 is a flowchart for describing an example of the operation of the gait measurement system 3. In the following description along the flowchart of FIG. 23, the gait measurement system 3 is an operation subject.

In FIG. 23, first, the gait measurement system 3 measures acceleration and angular velocity (step S31).

Next, the gait measurement system 3 calculates a walking parameter using at least one of acceleration data and angular velocity data (step S32).

Next, the gait measurement system 3 generates time-series data of walking parameters for several steps (step S33).

Next, the gait measurement system 3 calculates the symmetry of the walking parameters using the time-series data of the walking parameter (step S34).

Next, the gait measurement system 3 calculates the symmetry of the step lengths by applying the calculated symmetry of the walking parameters to the regression model (step S35).

Next, the gait measurement system 3 calculates the step lengths of the left and right feet using the calculated symmetry of the step lengths (step S36).

Then, the gait measurement system 3 displays the information on the symmetry of walking such as the step lengths of the left and right feet or the symmetry of the step lengths on the display unit 330 of the display device 33 (step S37).

An example of the operation of the gait measurement system 3 of the present example embodiment has been described above. The flowchart of FIG. 23 is an example, and the operation of the gait measurement system 3 of the present example embodiment is not limited to the processing along the flowchart of FIG. 23.

### Modified Example

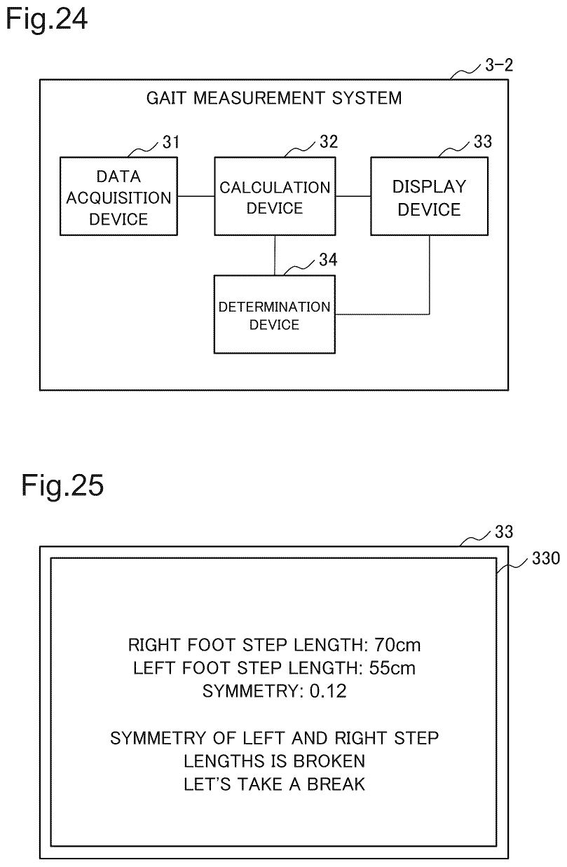

Next, a modified example of the present example embodiment will be described with reference to the drawings. FIG. 24 is a block diagram illustrating an example of a configuration of a gait measurement system 3-2 according to the modified example. The gait measurement system 3-2 of FIG. 24 is different from the gait measurement system 3 of FIG. 21 in that the gait measurement system 3-2 includes a determination device 34. Configurations of each of a data acquisition device 31, a calculation device 32, and a display device 33 of the gait measurement system 3-2 in FIG. 24 are similar to the configurations of the gait measurement system 3 in FIG. 21, and thus a detailed description thereof will be omitted.

The determination device 34 is connected to the calculation device 32 and the display device 33. The determination device 34 acquires information on step lengths of left and right feet or a symmetry of step lengths from the calculation device 32. The determination device 34 determines values of the step lengths of the left and right feet or values of the symmetry of the step lengths according to a magnitude relationship with a preset threshold. The determination device 34 outputs, to the display device 33, determination results related to the values of the step lengths of the left and right feet or the values of the symmetry of the step lengths. The determination results regarding the values of the step lengths of the left and right feet and the values of the symmetry of the step lengths are displayed on the display unit 330 of the display device 33.

For example, the determination device 34 makes a determination regarding energy cost, pain, muscle weakness, a degree of recovery from stroke due to rehabilitation, and the like of a pedestrian according to the magnitude relationship with the preset threshold value or a difference from the threshold value. For example, a plurality of threshold values may be set, and the determination results may be prepared for each area determined by the plurality of threshold values. The determination device 34 generates the display information according to the relationship between the determination result and the threshold value, and outputs the display information to display device 33.

FIG. 25 illustrates an example in which the values of the step lengths of the left and right feet, the value of the symmetry of the step lengths, and the determination results are displayed on the display unit 330 of the display device 33 as the information on the step lengths of the left and right feet and the symmetry of the step lengths. The example of FIG. 25 is an example in which information indicating that the step length of the right foot is 70 cm, the step length of the left foot is 55 cm, and the symmetry of the left and right feet is 0.12 is displayed on the display unit 330 of the display device 33. Furthermore, in the example of FIG. 25, based on the value of the symmetry, a determination result of “the symmetry of the left and right step lengths has collapsed” or an advice of “Let's take a little break” according to the determination result is displayed on the display unit 330.

As illustrated in FIG. 25, a user who visually recognizes the information displayed on the display unit 330 of the display device 33 can estimate a walking state of a pedestrian according to the information displayed on the display unit 330. The information displayed on the display unit 330 is not limited to the example of FIG. 25 as long as the information is relevant to the step lengths of the left and right feet and the symmetry of the step lengths.

As described above, the gait measurement system of the present example embodiment includes the display device that displays the information on the symmetry of walking. According to the present example embodiment, the walking state of the pedestrian can be estimated by referring to the information on the symmetry of walking displayed on the display device.

### Hardware

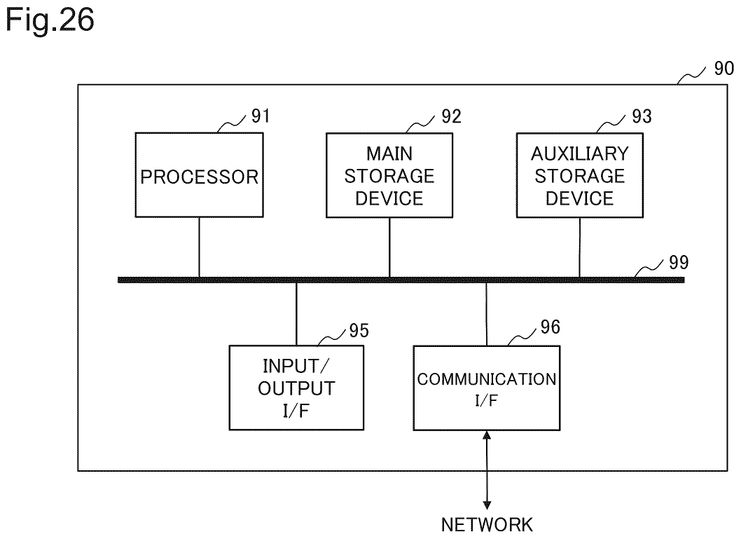

Here, the hardware configuration for achieving the calculation device according to each example embodiment of the present invention will be described by taking the information processing device 90 (also referred to as a computer) of FIG. 26 as an example. The information processing device 90 in FIG. 26 is a configuration example for executing the processing of the calculation device and the like of each example embodiment, and does not limit the scope of the present invention.

As illustrated in FIG. 26, the information processing device 90 includes a processor 91, a main storage device 92, an auxiliary storage device 93, an input/output interface 95, and a communication interface 96. In FIG. 26, the interface is abbreviated as I/F (interface). The processor 91, the main storage device 92, the auxiliary storage device 93, the input/output interface 95, and the communication interface 96 are data-communicably connected to each other via a bus 99. In addition, the processor 91, the main storage device 92, the auxiliary storage device 93, and the input/output interface 95 are connected to a network such as the Internet or an intranet via the communication interface 96.

The processor 91 expands the program stored in the auxiliary storage device 93 or the like to the main storage device 92, and executes the expanded program. In the present example embodiment, a software program installed in the information processing device 90 may be used. The processor 91 executes the processing by the calculation device according to the present example embodiment.

The main storage device 92 has an area in which the program is developed. The main storage device 92 is achieved by, for example, a volatile memory such as a dynamic random access memory (DRAM). Furthermore, a nonvolatile memory such as a magnetoresistive random access memory (MRAM) may be configured/added as the main storage device 92.

The auxiliary storage device 93 stores various data. The auxiliary storage device 93 is configured by a local disk such as a hard disk or a flash memory. Various data may be stored in the main storage device 92, and the auxiliary storage device 93 may be omitted.

The input/output interface 95 is an interface for connecting the information processing device 90 and peripheral devices. The communication interface 96 is an interface for connecting to an external system or device through a network such as the Internet or an intranet based on a standard or a specification. The input/output interface 95 and the communication interface 96 may be shared as an interface connected to an external device.

The information processing device 90 may be configured to connect input devices such as a keyboard, a mouse, or a touch panel, if necessary. These input devices are used to input information and settings. When a touch panel is used as the input device, a display screen of the display device may also serve as the interface of the input device. Data communication between the processor 91 and the input device may be mediated by the input/output interface 95.

In addition, the information processing device 90 may be provided with the display device for displaying information. When the display device is provided, the information processing device 90 preferably includes a display control device (not illustrated) for controlling the display of the display device. The display device may be connected to the information processing device 90 via the input/output interface 95.

The information processing device 90 may be equipped with a disk drive, if necessary. The disk drive is connected to the bus 99. The disk drive mediates reading a data/program from the recording medium, writing the processing result of the information processing device 90 to the recording medium, and the like between the processor 91 and a recording medium (program recording medium) (not illustrated). The recording medium can be achieved by, for example, an optical recording medium such as a compact disc (CD) or a digital versatile disc (DVD). Further, the recording medium may be achieved by a semiconductor recording medium such as a universal serial bus (USB) memory or a secure digital (SD) card, a magnetic recording medium such as a flexible disk, or other recording media.

The above is an example of a hardware configuration for achieving the calculation device according to each example embodiment of the present invention. The hardware configuration of FIG. 26 is an example of a hardware configuration for achieving the calculation device according to each example embodiment, and does not limit the scope of the present invention. In addition, the program for causing a computer to execute the processing related to the calculation device according to each example embodiment is also included in the scope of the present invention. Furthermore, the program recording medium in which the program according to each example embodiment is recorded is also included in the scope of the present invention.

The components of the calculation device of each example embodiment may be arbitrarily combined. In addition, the components such as the calculation device of each example embodiment may be achieved by software or may be achieved by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

### REFERENCE SIGNS LIST

1, 2, 3 gait measurement system
11, 21, 31 data acquisition device
12, 22, 32 calculation device
33 display device
34 determination device
111 acceleration sensor
112 angular velocity sensor
113 signal processing unit
115 data transmission unit
121, 221 walking parameter calculation unit
123, 223 symmetry calculation unit
225 storage unit
227 step length calculation unit
330 display unit

What is claimed is:

1. A gait measurement system, comprising:
a data acquisition device configured to measure physical quantities related to movements of left and right feet; and
a calculation device including
at least one memory storing instructions, and
at least one processor connected to the at least one memory and configured to execute the instructions to calculate a symmetry of walking using the physical quantities related to the movements of the left and right feet.

2. The gait measurement system according to claim 1, wherein
the at least one processor of the calculation device is configured to execute the instructions to:
generate time-series data of walking parameters by using the physical quantities related to the movements of the left and right feet; and
calculate the symmetry of the walking parameters of the left and right feet as the symmetry of walking using the time-series data of walking parameters of the left and right feet.

3. The gait measurement system according to claim 2, wherein
the data acquisition device is configured to
measure at least one of acceleration in a triaxial direction and angular velocity in the triaxial direction as the physical quantities related to the movements of the left and right feet,
the at least one processor of the calculation device is configured to execute the instructions to
generate time-series data of attitude angles of the left and right feet by using at least one of the acceleration in the triaxial direction and the angular velocity in the triaxial direction measured by the data acquisition device, and
calculate the symmetry of the walking parameters by using maximum values of peaks appearing in the time-series data of the attitude angles of the left and right feet.

4. The gait measurement system according to claim 3, wherein
the at least one processor of the calculation device is configured to execute the instructions to
calculate the symmetry of the walking parameters by using a maximum value at a time when a dorsal flexion angle turns to be maximum among the maximum values of the peaks appearing in the time-series data of the attitude angles of the left and right feet.

5. The gait measurement system according to claim 2, wherein
the data acquisition device is configured to
measure at least one of acceleration in the triaxial direction and angular velocity in the triaxial direction as the physical quantities related to the movements of the left and right feet,
the at least one processor of the calculation device is configured to execute the instructions to
generate time-series data of sensor heights of the left and right feet by using at least one of the acceleration in the triaxial direction and the angular velocity in the triaxial direction measured by the data acquisition device, and
calculate the symmetry of the walking parameters by using maximum values of peaks appearing in the time-series data of the sensor heights of the left and right feet.

6. The gait measurement system according to claim 5, wherein
the at least one processor of the calculation device is configure to execute the instructions to
calculate the symmetry of the walking parameters by using a maximum value at a time when a dorsal flexion angle turns to be maximum immediately before a heel of foot swung out forward is landed, among the maximum values of the peaks appearing in the time-series data of the sensor heights of the left and right feet.

7. The gait measurement system according to claim 2, wherein
the at least one processor of the calculation device is configured to execute the instructions to:
store a regression model in which the symmetry of the walking parameters and a symmetry of step lengths are associated with each other; and
calculate the symmetry of the step lengths from the symmetry of the walking parameters using the regression model and calculate the step lengths of the left and right feet using the calculated symmetry of the step lengths.

8. The gait measurement system according to claim 1 further comprising:
a display device configured to display information on the symmetry of walking.

9. A gait measurement method, comprising:
by a computer,
acquiring physical quantities related to movements of left and right feet; and
calculating a symmetry of walking using the acquired physical quantities related to the movements of the left and right feet.

10. A non-transitory program recording medium recorded with a program causing a computer to perform a process comprising:
acquiring physical quantities related to movements of left and right feet; and
calculating a symmetry of walking using the acquired physical quantities related to the movements of the left and right feet.
